SEQUENCE LISTING (47 sequences)

SEQ ID NO: 1 - DNA, 2556 bp, Artificial Sequence
  TGF beta RIII nucleotide sequence
atgacttcccattatgtgattgccatctttgccctgatgagctcctgtttagccactgcaggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtgcaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgcagaggagatgggagatgaggaagtccacactattcctcctgagctacggatcctgctggaccctggtgccctgcctgccctgcagaacccgcccatccggggaggggaaggccaaaatggaggccttccgtttcctttcccagatatttccaggagagtctggaatgaagagggagaagatgggctccctcggccaaaggaccctgtcattcccagcatacaactgtttcctggtctcagagagccagaagaggtgcaagggagcgtggatattgccctgtctgtcaaatgtgacaatgagaagatgatcgtggctgtagaaaaagattcttttcaggccagtggctactcggggatggacgtcaccctgttggatcctacctgcaaggccaagatgaatggcacacactttgttttggagtctcctctgaatggctgcggtactcggccccggtggtcagcccttgatggtgtggtctactataactccattgtgatacaggttccagcccttggggacagtagtggttggccagatggttatgaagatctggagtcaggtgataatggatttccgggagatatggatgaaggagatgcttccctgttcacccgacctgaaatcgtggtgtttaattgcagccttcagcaggtgaggaaccccagcagcttccaggaacagccccacggaaacatcaccttcaacatggagctatacaacactgacctctttttggtgccctcccagggcgtcttctctgtgccagagaatggacacgtttatgttgaggtatctgttactaaggctgaacaagaactgggatttgccatccaaacgtgctttatctctccatattcgaaccctgataggatgtctcattacaccattattgagaatatttgtcctaaagatgaatctgtgaaattctacagtcccaagagagtgcactttcctatcccgcaagctgacatggataagaagcgattcagctttgtcttcaagcctgtcttcaacacctcactgctctttctacagtgtgagctgacgctgtgtacgaagatggagaagcacccccagaagttgcctaagtgtgtgcctcctgacgaagcctgcacctcgctggacgcctcgataatctgggccatgatgcagaataagaagacgttcactaagccccttgctgtgatccaccatgaagcagaatctaaagaaaaaggtccaagcatgaaggaaccaaatccaatttctccaccaattttccatggtctggacaccctaaccgtgatgggcattgcgtttgcagcctttgtgatcggagcactcctgacgggggccttgtggtacatctattctcacacaggggagacagcaggaaggcagcaagtccccacctccccgccagcctcggaaaacagcagtgctgcccacagcatcggcagcacgcagagcacgccttgctccagcagcagcacggcctag

SEQ ID NO: 2 - DNA, 24 bp, Artificial Sequence
  TGF beta RIII sense primer
accgtgatgggcattgcgtttgca

SEQ ID NO: 3 - DNA, 25 bp, Artificial Sequence
  TGF beta RIII antisense primer
gtgctctgcgtgctgccgatgctgt

SEQ ID NO: 4 - DNA, 20 bp, Artificial Sequence
  GAPDH F primer
accaggtggtctcctctgac

SEQ ID NO: 5 - DNA, 20 bp, Artificial Sequence
  GAPDH R primer
tgctgtagccaaattcgttg

SEQ ID NO: 6 - DNA, 19 bp, Artificial Sequence
  E2F
ctgaagtgactggacgaga

SEQ ID NO: 7 - DNA, 20 bp, Artificial Sequence
  E2R
cctgggtaacagagtgaaac

SEQ ID NO: 8 - DNA, 20 bp, Artificial Sequence
  E3-1F
tgatcacccttgcccctttg

SEQ ID NO: 9 - DNA, 20 bp, Artificial Sequence
  E3-1R
tgcagtgcggagattcagga

SEQ ID NO: 10 - DNA, 17 bp, Artificial Sequence
  E3-2F
gttttgtcaggctgtgc

SEQ ID NO: 11 - DNA, 18 bp, Artificial Sequence
  E3-2R
gttgaaccccagaagaga

SEQ ID NO: 12 - DNA, 19 bp, Artificial Sequence
  E4F
tttctgccctctttctgtt

SEQ ID NO: 13 - DNA, 21 bp, Artificial Sequence
  E4R
ccattatgtccttgtgctaag

SEQ ID NO: 14 - DNA, 18 bp, Artificial Sequence
  E5-1F
aggttcgatttacaagca

SEQ ID NO: 15 - DNA, 21 bp, Artificial Sequence
  E5-1R
ttaacagatgttcvatttcca

SEQ ID NO: 16 - DNA, 18 bp, Artificial Sequence
  E5-2F
cagcaaacttctccttga

SEQ ID NO: 17 - DNA, 20 bp, Artificial Sequence
  E5-2R
attgcctgtcataaatcagt

SEQ ID NO: 18 - DNA, 20 bp, Artificial Sequence
  E6F
cctcagtggtttgacagatt

SEQ ID NO: 19 - DNA, 21 bp, Artificial Sequence
  E6R
tcatctcttgtcacactcaca

SEQ ID NO: 20 - DNA, 20 bp, Artificial Sequence
  E7F
aactttctggcatgtaggtc

SEQ ID NO: 21 - DNA, 18 bp, Artificial Sequence
  E7R
gacatgctccaccaactt

SEQ ID NO: 22 - DNA, 22 bp, Artificial Sequence
  E8-1F
attttagactcatgagtgattt

SEQ ID NO: 23 - DNA, 20 bp, Artificial Sequence
  E8-1R
gccattgtgtatgaagttat

SEQ ID NO: 24 - DNA, 20 bp, Artificial Sequence
  E8-2F
ccaaatcaataagagatgac

SEQ ID NO: 25 - DNA, 19 bp, Artificial Sequence
  E8-2R
gaaatgacagttcctcact

SEQ ID NO: 26 - DNA, 18 bp, Artificial Sequence
  E9-1F
ggcctggcatcaaacact

SEQ ID NO: 27 - DNA, 20 bp, Artificial Sequence
  E9-1R
gagcccatcttctccctctt

SEQ ID NO: 28 - DNA, 18 bp, Artificial Sequence
  E9-2F
ccgtttcctttcccagat

SEQ ID NO: 29 - DNA, 19 bp, Artificial Sequence
  E9-2R
tagcctctcttccctcctg

SEQ ID NO: 30 - DNA, 17 bp, Artificial Sequence
  E10F
acagaactgcctgrggg

SEQ ID NO: 31 - DNA, 20 bp, Artificial Sequence
  E10R
caaagctttgttctggaaaa

SEQ ID NO: 32 - DNA, 17 bp, Artificial Sequence
  E11F
aggcagaaccaaacaca

SEQ ID NO: 33 - DNA, 20 bp, Artificial Sequence
  E11R
accccctactgataacaaac

SEQ ID NO: 34 - DNA, 19 bp, Artificial Sequence
  E12F
cctgtgggttgttatttcc

SEQ ID NO: 35 - DNA, 21 bp, Artificial Sequence
  E12R
aaggtcaaggctaactttcag

SEQ ID NO: 36 - DNA, 20 bp, Artificial Sequence
  E13-1F
ttgtgcctaaagtgaaagtg

SEQ ID NO: 37 - DNA, 16 bp, Artificial Sequence
  E13-1R
tcagcttgcgggatag

SEQ ID NO: 38 - DNA, 20 bp, Artificial Sequence
  E13-2F
gaaattctavagtcccaaga

SEQ ID NO: 39 - DNA, 20 bp, Artificial Sequence
  E13-2R
ctaaaaatgccaaaataacc

SEQ ID NO: 40 - DNA, 19 bp, Artificial Sequence
  E14F
ccctgattctgrgctttgt

SEQ ID NO: 41 - DNA, 16 bp, Artificial Sequence
  E14R
tctgatcgtgcctccc

SEQ ID NO: 42 - DNA, 20 bp, Artificial Sequence
  E15F
gtttctgctgagactttgat

SEQ ID NO: 43 - DNA, 17 bp, Artificial Sequence
  E15R
ccctggaggtttatttc

SEQ ID NO: 44 - DNA, 19 bp, Artificial Sequence
  E16F
tgatgcagactaaccaaaa

SEQ ID NO: 45 - DNA, 20 bp, Artificial Sequence
  E16R
aagctgttcaccaactctta

SEQ ID NO: 46 - DNA, 20 bp, Artificial Sequence
  E17F
tgcgtctttctctgactctg

SEQ ID NO: 47 - DNA, 18 bp, Artificial Sequence
  E17R
tggcagtagctgagctga

(19) United States
(12) Patent Application Publication
(10) Pub. No.: US 2011/0224090 A1
(43) Pub. Date: Sep. 15, 2011

(54) SINGLE NUCLEOTIDE POLYMORPHIC MARKERS OF TGFBetaRIII GENE FOR DIAGONISIS OF HEPATOCELLULAR CARCINOMA

(76) Inventors: Suk Woo NAM, Seoul (KR); Hyun Jin Bae, Seoul (KR)

(21) Appl. No.: 12/884,099

(22) Filed: Sep. 16, 2010

(30) Foreign Application Priority Data
Mar. 12, 2010 (KR) 10-2010-0022112

(51) Int. Cl. C40B 30/04; C07H 21/04; C40B 40/06; G01N 33/50; C12Q 1/68

(52) U.S. Cl. 506/9; 536/23.5; 536/24.33; 506/16; 436/94; 435/6.12; 435/6.14

(57) ABSTRACT

The present invention relates to diagnostic markers for hepatocellar carcinoma and a method for predicting and diagnosing susceptibility to hepatocellular carcinoma, and more particularly, to polymorphic markers for the diagnosis of hepatocellular carcinoma based on polymorphisms present in exons of the TGFβRIII gene represented by SEQ ID No. 1, a diagnostic composition for hepatocellular carcinoma using the same, a diagnostic kit, a microarray, and a method for diagnosing hepatocellular carcinoma. The polymorphic markers for the diagnosis of hepatocellular carcinoma according to the present invention are genetic markers useful to diagnose genetic susceptibility specific to hepatocellular carcinoma. With these markers, susceptibility to hepatocellular carcinoma can be comprehensively determined.

### TECHNICAL FIELD

The present invention relates to single nucleotide polymorphic markers of the TGFβRIII gene for the diagnosis of hepatocellular carcinoma, and more particularly, to markers for the diagnosis of hepatocellular carcinoma based on polymorphisms of the TGFβRIII gene.

### BACKGROUND ART

Hepatocellular carcinoma (HCC) is the fifth most common cancer in the world, responsible for 500,000 deaths globally every year (Okuda 2000). However, the overall survival of patients with HCC has not improved over the last 20 years, with the incidence rate almost equal to the death rate (Marrero, Fontana et al. 2005). The known major risk factors for HCC are chronic hepatitis resulting from infection with hepatitis B virus or hepatitis C virus and exposure to carcinogens such as aflatoxin B1 (Thorgeirsson and Grisham 2002).

Even if the risk factors for hepatocellular carcinoma are associated with persistent infection with hepatitis B virus or hepatitis C virus, the molecular mechanism in hepatocellular carcinoma cells have not been fully elucidated yet. Previous studies have reported that genes, such as altered p53, β-catenin, AXINI, p21 (WAF1/CIPI), p27 Kip, etc., are involved in hepatocarcinogenesis.

However, these individual genetic changes do not precisely reflect the heterogenous nature and clinical characteristics of HCC patients. The cellular and molecular diversities within individual HCC are demanding new approaches, in addition to the existing genetic studies.

In association with this, microarray technology is a new technique that allows simultaneous measurement of tens of thousands of gene expressions in a single experiment beyond the measurement range of a single gene. Microarray technology has been applied to most of the studies on cancers including hepatocellular carcinoma, and allows the diagnosis, prognosis, and prediction of cancer at the molecular level by extracting genes actively involved in the development and progression of cancer. Hepatocellular carcinoma has been diagnosed by conducting a tissue examination or testing hepatocellular carcinoma marker proteins, such as alpha-fetoprotein (hereinafter, “AFP”).

At present, the best-known biomarkers for the diagnosis, prognosis, and therapy evaluation associated with hepatocellular carcinoma include AFP, PIVKA (Protein Induced by Vitamin K Absence)-II, etc., which are lack in specificity and sensitivity. The usefulness of AFP in the diagnosis of hepatocellular carcinoma is well known. As well as the diagnosis of progressed hepatocellular carcinoma, periodic AFP measurement for early detection of hepatocellular carcinoma is required because there are about 3-10% of liver cirrhosis patients who are reported to develop hepatocellular carcinoma during the natural course of liver cirrhosis. However, AFP increases at high concentrations in positive diseases, such as alcoholic hepatitis, chronic hepatitis, or liver cirrhosis, as well as in hepatocellular carcinoma, AFP turns out to be false positive in many cases, and the actual positive rate of AFP is no more than 50-60% (sensitivity is 29.9% and 65.8% in 20 ng/ml and 400 ng/ml, respectively).

PIVKA-II is DCP (des-r-carboxyprothrombin), i.e., an abnormal prothrombin devoid of coagulation activity, and has been reported to have a sensitivity of 48.2% and a specificity of 95.9% in the diagnosis of hepatocellular carcinoma independently from serum AFP. These biological indices are clinically used at present, but do not reflect all of the biological characteristics of hepatocellular carcinoma and are of limited use. Therefore, the discovery of markers that can diagnose hepatocellular carcinoma more specifically and effectively than the current AFP and PIVKAII, and the development of a test reagent for early diagnosis of hepatocellular carcinoma using the markers are in demand.

### DISCLOSURE

### Technical Problem

The present inventors have completed the present invention upon discovering the genetic association between the TGFβRIII gene and hepatocellular carcinoma for the first time, finding that six single nucleotide polymorphisms are present in exons of the TGFβRIII gene, and discovering that these six single nucleotide polymorphisms can be used as markers for the diagnosis of hepatocellular carcinoma.

Accordingly, it is an object of the present invention to provide single nucleotide polymorphic markers of the TGFβRIII gene for the diagnosis of hepatocellular carcinoma, and more particularly, to markers for the diagnosis of hepatocellular carcinoma based on polymorphisms of the TGFβRIII gene.

It is another object of the present invention to provide a diagnostic composition for hepatocellular carcinoma, comprising a primer for detecting polymorphic markers of the TGFβRIII gene, and a diagnostic kit for hepatocellular carcinoma comprising the composition.

It is still another object of the present invention to provide a microarray for the diagnosis of hepatocellular carcinoma, comprising a polynucleotide comprising a polymorphic site of the TGFβRIII gene capable of diagnosing hepataocelular carcinoma.

It is yet another object of the present invention to provide a method for detecting polymorphisms of the TGFβRIII gene for the diagnosis of hepatocellular carcinoma, which can provide information required for the diagnosis of hepatocellular carcinoma.

It is a further object of the present invention to provide a method for predicting or diagnosing hepatocellular carcinoma using single nucleotide polymorphic markers of the TGFβRIII gene for the diagnosis of hepatocellular carcinoma according to the present invention.

### Technical Solution

To accomplish the aforementioned objects of the present invention, there is provided a polymorphic marker for the diagnosis of hepatocellular carcinoma, comprising one or more polynucleotides, which are TGFβRIII (transforming growth factor receptor III) polynucleotides represented by SEQ ID No. 1, selected from the group consisting of: a polynucleotide comprising contiguous 20 to 100 DNA sequences with C or T at the 44^{th} base of SEQ ID No. 1; a polynucleotide comprising contiguous 20 to 100 DNA sequences with A or G at the 216^{th} base of SEQ ID No. 1; a polynucleotide comprising contiguous 20 to 100 DNA sequences with A or G at the 519^{th} base of SEQ ID No. 1; a polynucleotide comprising contiguous 20 to 100 DNA sequences with T or C at the 2028^{th} base of SEQ ID No. 1; a polynucleotide comprising contiguous 20 to 100 DNA sequences with G or A at the 2133^{th} base of SEQ ID No. 1; a polynucleotide comprising contiguous 20 to 100 DNA sequences with T or C at the 2247^{th} base of SEQ ID No. 1; and a complementary polynucleotide thereof.

In one embodiment of the present invention, the 44^{th} base of SEQ ID No. 1 is located in exon 2 of the TGFβRIII gene, the 216^{th} base of SEQ ID No. 1 is located in exon 3 of the TGFβRIII gene, the 519^{th} base of SEQ ID No. 1 is located in exon 5 of the TGFβRIII gene, the 2028^{th} and 2123th bases of SEQ ID No. 1 are located in exon 13 of the TGFβRIII gene, and the 2247^{th} base of SEQ ID No. 1 is located in exon 14 of the TGFβRIII gene.

In one embodiment of the present invention, the hepatocellular carcinoma may be human hepatocellular carcinoma.

Furthermore, the present invention provides a diagnostic composition for hepatocellular carcinoma, the composition comprising a primer for amplifying a polynucleotide, which is a TGFβRIII (transforming growth factor receptor III) polynucleotide represented by SEQ ID No. 1, the polynucleotide comprising: a polymorphic site of the 44^{th} base of SEQ ID No. 1; a polymorphic site of the 216^{th} base of SEQ ID No. 1; a polymorphic site of the 519^{th} base of SEQ ID No. 1; a polymorphic site of the 2028^{th} base of SEQ ID No. 1; a polymorphic site of the 2133^{th} base of SEQ ID No. 1; or a polymorphic site of the 2247^{th} base of SEQ ID No. 1.

In one embodiment of the present invention, the primer may be selected from the group consisting of: a primer pair represented by SEQ ID Nos. 6 and 7 for detecting a polymorphic site of the 44^{th} base of SEQ ID No. 1; a primer pair represented by SEQ ID Nos. 8 and 9 or SEQ ID Nos. 10 and 11 for detecting a polymorphic site of the 216^{th} base of SEQ ID No. 1; a primer pair represented by SEQ ID Nos. and 15 or SEQ ID Nos. 16 and 17 for detecting a polymorphic site of the 519^{th} base of SEQ ID No. 1; a primer pair represented by SEQ ID Nos. 36 and 37 for detecting a polymorphic site of the 2028^{th} base of SEQ ID No. 1; a primer pair represented by SEQ ID Nos. 38 and 39 for detecting a polymorphic site of the 2133^{th} base of SEQ ID No. 1; and a primer pair represented by SEQ ID Nos. 40 and 41 for detecting a polymorphic site of the 2247^{th} base of SEQ ID No. 1.

Furthermore, the present invention provides a microarray for the diagnosis of hepatocellular carcinoma, comprising a polynucleotide, which is the diagnostic marker for hepatocellular carcinoma according to the present invention.

Furthermore, the present invention provides a diagnostic kit for hepatocellular carcinoma comprising the diagnostic composition for hepatocellular carcinoma according to the present invention.

Furthermore, in order to provide information required for the diagnosis of hepatocellular carcinoma, the present invention provides a method for detecting a polymorphism (44C/T) of the 44^{th} base of SEQ ID No. 1, a polymorphism (216A/G) of the 216^{th} base of SEQ ID No. 1, a polymorphism (519A/G) of the 519^{th} base of SEQ ID No. 1, a polymorphism (2028T/C) of the 2028^{th} base of SEQ ID No. 1, a polymorphism (2133G/A) of the 2133^{th} base of SEQ ID No. 1, or a polymorphism (2247T/C) of the 2247^{th} base of SEQ ID No. from a TGFβRIII (transforming growth factor receptor III) in a polynucleotide represented by SEQ ID No. 1 by base sequence analysis from patient samples.

In one embodiment of the present invention, the base sequence analysis may be performed by sequencing analysis, hybridization by microarray, allele specific PCR, dynamic allele-specific hybridization (DASH), PCR extension assay, or TaqMan technique.

Furthermore, the present invention provides a method for predicting or diagnosing hepatocellular carcinoma, the method comprising the steps of: obtaining a nucleic acid sample from a specimen; amplifying one or more polymorphic sites selected from the group consisting of the 44^{th} base, 216^{th} base, 519^{th} base, 2028^{th} base, 2133th base, and 2247^{th} base of SEQ ID No. 1 of a TGFβRIII (transforming growth factor receptor III) polynucleotide represented by SEQ ID No. 1; and determining by analysis of the amplified DNA sequence whether the 44^{th} base of the polynucleotide of SEQ ID No. 1 is C or T, whether the 216^{th} base of the polynucleotide of SEQ ID No. 1 is A or G, whether the 519^{th} base of the polynucleotide of SEQ ID No. 1 is A or G, whether the 2028^{th} base of the polynucleotide of SEQ ID No. 1 is T or C, whether the 2133^{th} base of the polynucleotide of SEQ ID No. 1 is G or A, or whether the 2247^{th} base of the polynucleotide of SEQ ID No. 1 is T or C.

In one embodiment of the present invention, the amplification of a polymorphic site may be performed using a primer pair selected from the group consisting of: a primer pair represented by SEQ ID Nos. 6 and 7 capable of detecting a polymorphic site of the 44^{th} base of SEQ ID No. 1; a primer pair represented by SEQ ID Nos. 8 and 9 or SEQ ID Nos. 10 and 11 capable of detecting a polymorphic site of the 216^{th} base of SEQ ID No. 1; a primer pair represented by SEQ ID Nos. 14 and 15 or SEQ ID Nos. 16 and 17 capable of detecting a polymorphic site of the 519^{th} base of SEQ ID No. 1; a primer pair represented by SEQ ID Nos. 36 and 37 capable of detecting a polymorphic site of the 2028^{th} base of SEQ ID No. 1; a primer pair represented by SEQ ID Nos. 38 and 39 capable of detecting a polymorphic site of the 2133^{th} base of SEQ ID No. 1; and a primer pair represented by SEQ ID Nos. 40 and 41 capable of detecting a polymorphic site of the 2247^{th} base of SEQ ID No. 1.

In one embodiment of the present invention, the method may further comprise the step of determining that the risk of developing hepatocellular carcinoma is high if, in the polynucleotide of SEQ ID No. 1, the 44^{th} base is T, the 216^{th} base is G, the 519^{th} base is A, the 2028^{th} base is T, the 2133th base is G, or the 2247^{th} base is C.

### Advantageous Effects

The polymorphic marker for the diagnosis of hepatocellular carcinoma according to the present invention can be used as a genetic maker useful to diagnose genetic susceptibility specific to hepatocellular carcinoma, and the marker based on the polymorphisms of the TGFβRIII gene according to the present invention is useful to effectively predict and diagnose susceptibility to hepatocellular carcinoma at an early stage.

### DESCRIPTION OF DRAWINGS

The above and other objects and features of the present invention will become apparent from the following description of the preferred embodiments given in conjunction with the accompanying drawings, in which:

FIG. 1 shows a heat-map of the differential expressions of TGFβ and TGFβ receptors in a dysplastic HCC nodule;

FIG. 2 shows a result of qRT-PCR analysis of TGFβRIII in HCC;

FIG. 3 shows results of the loss of heterozygosity with microsatellite markers D1S188, D1S406, D1S435, and D1S2804 (N: normal; T: tumor; arrows: LOH); and

FIGS. 4a and 4b show six polymorphisms present in exons in the TGFβRIII gene sequence according to the present invention.

### BEST MODE FOR THE INVENTION

First, the terms used in the present invention are defined as follows.

In the present invention, the term “genetic polymorphism” refers to genetic variations occurring at a frequency of at least 1% in a given population. The insertion, deletion, or substitution of one nucleotide in DNA is referred to as a single nucleotide polymorphism (SNP). If changes in base sequence caused by the SNP result in amino acid changes, this is referred to as a nonsynonymous SNP, and if they result in no amino acid changes, this is referred to as a silent SNP or synonymous SNP.

The term “marker” refers to a nucleotide sequence or encoded product thereof (e.g., a protein) used as a point of reference when identifying a locus or a linked locus. A marker can be derived from genomic nucleotide sequences or from expressed nucleotide sequences (e.g., from an RNA, an nRNA, an mRNA, cDNA, etc.), or from an encoded polypeptide. The term also refers to nucleic acid sequences complementary to or flanking the marker sequences, such as nucleic acids used as probes or primer pairs capable of amplifying the marker sequence.

The term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs (e.g. peptide nucleic acids) and as applicable to the embodiment being described, single (sense or antisense) and double-stranded polynucleotides. In the present invention, the terms “nucleic acid” and “nucleotide” are used interchangeably.

The term “primer” or “primers” refers to oligonucleotide sequences that hybridize to a complementary RNA or DNA target polynucleotide and serve as the starting points for the stepwise synthesis of a polynucleotide from mononucleotides by the action of a nucleotidyltransferase, as occurs for example in a polymerase chain reaction.

The term “allele factor” or “allele” denotes any of two or more alternative forms of a gene occupying the same chromosomal locus.

The term “allele frequency” refers to the frequency (proportion or percentage) at which an allele is present at a locus within an individual, within a line, or within a population of lines. One can estimate the allele frequency within a line or population by averaging the allele frequencies of a sample of individuals from that line or population.

The phrase “diseases and conditions associated with polymorphisms” refers to a variety of diseases or conditions, the susceptibility to which can be indicated in a subject based on the identification of one or more alleles.

The term “risk” refers to a statistically higher frequency of occurrence of the disease or condition in an individual carrying a particular polymorphic allele in comparison to the frequency of occurrence of the disease or condition in a member of a population that does not carry the particular polymorphic allele.

The term “phenotype” is a collection of morphological, physiological, or biochemical characteristics of an individual determined by genetic patterns. In a narrower sense, the term “phenotype” refers to alleles present on one gene.

By “subject” or “patient” is meant any single subject for which therapy is desired, including humans, cattle, dogs, guinea pigs, rabbits, chickens, insects and so on. Also intended to be included as a subject are any subjects involved in clinical research trials not showing any clinical sign of disease, or subjects involved in epidemiological studies, or subjects used as controls. In one embodiment of the present invention, the subject is a human.

By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; or cells from any time in gestation or development of the subject. The tissue sample may also be primary or cultured cells or cell lines.

Optionally, the tissue or cell sample is obtained from a primary or metastatic tumor. The tissue sample may contain compounds which are not naturally intermixed with the tissue in nature such as preservatives, anticoagulants, buffers, fixatives, nutrients, antibiotics, or the like. For the purposes herein, a “section” of a tissue sample is meant a single part or piece of a tissue sample, e.g. a thin slice of tissue or cells cut from a tissue sample. It is understood that multiple sections of tissue samples may be taken and subjected to analysis according to the present invention, provided that it is understood that the present invention comprises a method whereby the same section of tissue sample is analyzed at both morphological and molecular levels, or is analyzed with respect to both protein and nucleic acid.

The terms “cancer”, “tumor”, or “malignant” refer to or describe the physiological condition in mammals that is typically characterized by unregulated cell growth. Examples of cancer include but are not limited to, carcinoma, lymphoma, leukemia, blastoma, and sarcoma.

As used herein, the term “diagnosis” refers to the detection of a pathological state. For the purpose of the invention, the diagnosis is to confirm the development of hepatocellular carcinoma by detecting the expression of a diagnostic marker for hepatocellular carcinoma.

The term “a diagnostic marker, a marker for diagnosis, or a diagnosis marker”, as used herein, is intended to indicate a substance that can diagnose hepatocellular carcinoma by distinguishing hepatocellular carcinoma cells from normal cells, and includes organic biological molecules, quantities of which increase or decrease in hepatocellular carcinoma cells compared to normal cells, such as polypeptides or nucleic acids (e.g., mRNA, etc.), lipids, glycolipids, glycoproteins, and sugars (monosaccharides, disaccharides, oligosaccharides, etc.).

The term “treating”, as used herein, unless otherwise indicated, reversing, alleviating, or inhibiting the progress of, the disorder or condition to which such term applies, or one or more symptoms of such disorder or condition. The term “treatment”, as used herein, refers to the act of treating as “treating” is defined immediately above.

Hereinafter, the present invention will be described in detail.

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

Based on the result that there was a difference in expression level between the TGFβRIII gene in hepatocellular carcinoma tissue and its peripheral normal tissue, which was obtained while studying methods for more accurately and effectively diagnosing hepatocellular carcinoma, the present inventors identified the expression pattern of the TGFβRIII gene expressed in hepatocellular carcinoma tissue or hepatocellular carcinoma cell lines compared to normal tissue or cell lines, and assumed that polymorphisms of this gene can be used as markers for the diagnosis of hepatocellular carcinoma.

Hereupon, the present inventors found, for the first time, that, as a result of comparison in the expression pattern of the TGFβRIII gene between hepatocellular carcinoma tissue and the normal tissue, the expression of TGFβRIII in hepatocellular carcinoma tissue was down-regulated compared to the normal tissue, and further discovered polymorphisms of the TGFβRIII gene involved in hepatocarcinogenesis.

TGFβ is a multifunctional cytokine that regulates various cellular responses including cell proliferation, differentiation, migration, immunomodulation, etc., and is known to be expressed in various tissues, i.e., in almost every cell. Also, TGFβ controls the production of extracellular matrix proteins (ECM) by stimulating the synthesis of matrix components such as collagen and proteoglycans and inhibiting matrix degradation. Overexpression of TGFβ leads to excessive accumulation of ECM, thus stimulating fibrosis/sclerosis of an organ. In addition, in most human diseases associated with fibrosis/sclerosis, TGFβ overexpression has been observed.

Thus, chronic upregulation (overexpression) of TGFβ, which is the main cause of organ fibrosis, may cause, for example, diabetic nephropathy or other chronic renal diseases and the fibrosis of the liver, lungs, pancreas, and other organs.

Moreover, TGFβ has an immunosuppressive activity. The immunosuppressive effect of TGFβ is known to be partly derived from its antiproliferation activity, i.e., the inhibition of proliferation of T-lymphocytes and B-lymphocytes, and acts to regulate immune responses to tumors or infections. In particular, TGFβ plays a complex role in carcinogenesis, i.e., acts as a tumor suppressive factor in the initial stage and after that acts as a tumor inducing factor.

In many tumors, the plasma concentration of TGFβ1 correlates with the disease. In particular, TGFβ1 is able to stimulate tumor formation through vascularization and immunosuppressive activity because it is indirectly involved in vessel formation by upregulating the production of VEGF (Harmey et al. Ann. Surg. Oncol 5(3):271-278 (1998)).

In addition, there are three isoforms of TGFβ including TGFβ1, 2, and 3, which bind to specific receptors: TGFβRI; TGFβRII; and TGFβRIII, respectively. TGFβ activates the signaling system by binding to the respective receptors. Especially, TGFβRIII consisting of 849 amino acids is the most abundant TGFβ receptor.

According to the recently reported data, it was found that TGFβRIII is associated with the incidence of cancer. In particular, the expression of TGFβRIII in renal cell carcinoma was found to be down-regulated compared to normal cells, and down-regulation of TGFβRIII in breast, prostate, and non-small cell lung cancers was also confirmed.

However, the relationship between the degree of expression of TGFβRIII and hepatocellular carcinoma and polymorphisms of TGFβRIII specific to hepatocellular carcinoma have not been reported to date.

Accordingly, the present invention is characterized in that it provides polymorphic markers present in the TGFβRIII gene capable of diagnosing the development of hepatocelular carcinoma.

In one embodiment of the present invention, first, RNA was extracted from hepatocellular carcinoma tissue and normal tissue samples in order to analyze the differences in the degree of expression of the TGFβRIII gene between hepatocellular carcinoma cells and normal cells, and then RT-PCR was conducted by using a primer for amplifying the TGFβRIII gene to quantitatively analyze the expression amount of the gene.

As a result, it was observed that the expression of the TGFβRIII gene in the hepatocellular carcinoma tissue was significantly down-regulated compared to the normal tissue (see Example 2).

Additionally, the present inventors discovered 6 polymorphisms of the TGFβRIII gene present with a high frequency in hepatocellular carcinoma from the TGFβRIII gene whose expression was down-regulated specifically in the hepatocellular carcinoma tissue and cells. In other words, according to another embodiment of the present invention, the TGFβRIII gene was amplified by using a primer capable of covering the entire TGFβRIII gene of genomic DNA derived from hepatocellular carcinoma tissue and hepatocellular carcinoma cells, and polymorphisms of the TGFβRIII gene were investigated by SSCP analysis.

As a result, it was found that six polymorphisms were present in five exons (exons 2, 3, 5, 13, and 14) in the TGFβRIII gene, and that these six polymorphisms were present with a high frequency in hepatocellular carcinoma (see Example 3).

On the basis of these results, the present inventors confirmed that the TGFβRIII gene can be used as a diagnostic marker for hepatocellular carcinoma, and, further, that the six polymorphisms present in the TGFβRIII gene also can be used as diagnostic markers for hepatocellular carcinoma.

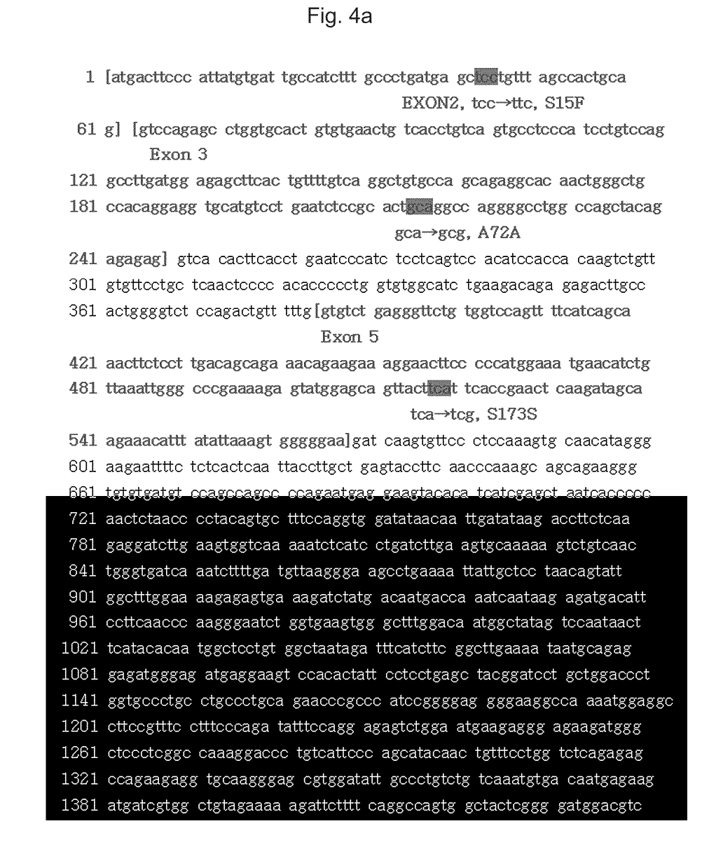

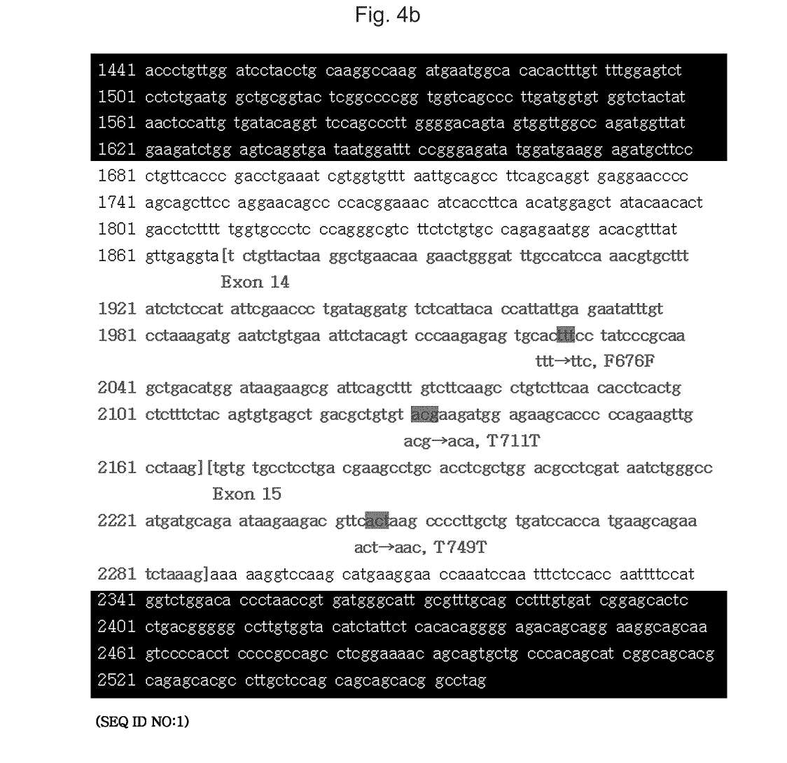

Additionally, in the present invention, the base sequence of the TGFβRIII gene is shown in SEQ ID NO. 1, and the six polymorphisms present in the TGFβRIII gene are as shown in the following table, and each of the polymorphic sites located in each of the exons (exons 2, 3, 5, 13, and 14) of the TGFβRIII gene represented by SEQ ID No. 1 are shown in FIGS. 4a and 4b.

[Polymorphic Markers For Diagnosis of Hepatocellular
Carcinoma According To The Present Invention]

| Exon | Base Position | Polymorphic Codon |
|---|---|---|
| 2 | 44^{th} base | TCC -> TTC |
| 3 | 216^{th} base | GCA -> GCG |
| 5 | 519^{th} base | TCA -> TCG |
| 13 | 2028^{th} base | TTT -> TTC |
| | 2133th base | ACG -> ACA |
| 14 | 2247^{th} base | ACT -> ACC |

Accordingly, the present invention provides a polymorphic marker for the diagnosis of hepatocellular carcinoma, comprising one or more polynucleotides, which are TGFβRIII (transforming growth factor receptor III) polynucleotides represented by SEQ ID No. 1, selected from the group consisting of: a polynucleotide comprising contiguous 20 to 100 DNA sequences with C or T at the 44^{th} base of SEQ ID No. 1; a polynucleotide comprising contiguous 20 to 100 DNA sequences with A or G at the 216^{th} base of SEQ ID No. 1; a polynucleotide comprising contiguous 20 to 100 DNA sequences with A or G at the 519^{th} base of SEQ ID No. 1; a polynucleotide comprising contiguous 20 to 100 DNA sequences with T or C at the 2028^{th} base of SEQ ID No. 1; a polynucleotide comprising contiguous 20 to 100 DNA sequences with G or A at the 2133^{th} base of SEQ ID No. 1; a polynucleotide comprising contiguous 20 to 100 DNA sequences with T or C at the 2247^{th} base of SEQ ID No. 1; and a complementary polynucleotide thereof.

The polymorphic marker for the diagnosis of hepatocellular carcinoma provided in the present invention can be used to detect susceptibility to hepatocellular carcinoma, particularly human hepatocellular carcinoma (HCC) at an early stage and aid in the prognosis, diagnosis, and treatment of patients with HCC.

In each of the polynucleotides comprising the polymorphic marker for the diagnosis of hepatocellular carcinoma, the 44^{th} base of SEQ ID No. 1 is located in exon 2 of the TGFβRIII gene, the 216^{th} base of SEQ ID No. 1 is located in exon 3 of the TGFβRIII gene, the 519^{th} base of SEQ ID No. 1 is located in exon 5 of the TGFβRIII gene, the 2028^{th} and 2123th bases of SEQ ID No. 1 are located in exon 13 of the TGFβRIII gene, and the 2247^{th} base of SEQ ID No. 1 is located in exon 14 of the TGFβRIII gene.

Therefore, polymorphic sites of the TGFβRIII gene included in the diagnostic marker for hepatocellular carcinoma according to the present invention may be present in the exons 2, 3, 5, 13, and 14 of the TGFβRIII gene, and, in the present invention, these polymorphic sites are named ‘TGFβRIII 44C/T’, ‘TGFβRIII 1216A/G’, ‘TGFβRIII 519A/G’, ‘TGFβRIII 2028T/C’, ‘TGFβRIII 2133G/A’, and ‘TGFβRIII 2247T/C’, respectively.

Moreover, as for the polymorphic marker usable for the diagnosis of hepatocellular carcinoma in the present invention, the marker may be a haplotype or diplotype polymorphic marker, more preferably, a haplotype polymorphic marker.

Further, as for the marker that can be used to diagnose hepatocellular carcinoma in the present invention, a polypeptide encoded by the polynucleotide, as well as the polynucleotide comprising a polymorphic site of the TGFβRIII gene revealed as discussed above in the present invention, can be used. Especially, amino acid sequences encoded by nucleotide sequences comprising a C or T allele at the 44^{th} base of the polynucleotide of SEQ ID No. 1 can be used as the polymorphic marker for the diagnosis of hepatocellular carcinoma of this invention. More specifically, if the 15^{th} amino acid of a TGFβRIII protein encoded by SEQ ID No. 1 is serine (S) or phenylalanine (F), a polypeptide comprising the amino acid can be used as a diagnostic marker for hepatocellular carcinoma.

The present invention provides a diagnostic composition for hepatocellular carcinoma, comprising primers for amplifying a polymorphic site of the TGFβRIII gene, the composition comprising a primer for amplifying a polynucleotide, the polynucleotide comprising: a polymorphic site of the 44^{th} base of SEQ ID No. 1; a polymorphic site of the 216^{th} base of SEQ ID No. 1; a polymorphic site of the 519^{th} base of SEQ ID No. 1; a polymorphic site of the 2028^{th} base of SEQ ID No. 1; a polymorphic site of the 2133th base of SEQ ID No. 1; and a polymorphic site of the 2247th base of SEQ ID No. 1.

The diagnostic composition of the present invention can be immobilized on a suitable carrier or support in order to enhance the rapidness and convenience of diagnosis (Antibodies: A Laboratory Manual, Harlow & Lane; Cold SpringHarbor, 1988). Examples of suitable carriers or supports include agarose, cellulose, nitrocellulose, dextran, Sephadex, Sepharose, liposomes, carboxymethyl cellulose, polyacrylamides, polystyrene, gabbros, filter paper, ion-exchange resin, plastic film, plastic tube, glass, polyamine-methyl vinyl-ether-maleic acid copolymer, amino acid copolymer, ethylene-maleic acid copolymer, nylon, cups, and flat packs.

Furthermore, the present invention provides a diagnostic kit for hepatocellular carcinoma comprising the diagnostic composition for hepatocellular carcinoma according to the present invention.

The diagnostic kit can include a reagent for polymerization, for example, dNTP, various polymerization enzymes, a colorizing agent, etc., in addition to the polynucleotide of the present invention, i.e., a polymorphic marker for the diagnosis of hepatocellular carcinoma.

The “primer for amplifying” refers to a single-strand oligonucleotide capable of initiating a template-directed DNA synthesis in an appropriate buffer under an appropriate condition (for example, in the presence of four different nucleoside triphosphates and a polymerizing agent such as DNA, RNA polymerase or reverse transcriptase) at a proper temperature. The length of the primer may vary according to the purpose of use, but is usually 10 to 30 nucleotides. A short primer molecule generally requires lower temperatures to be stably hybridized with a template. The primer sequence does not necessarily need to be completely complementary with the template, but should be sufficiently complementary to be hybridized with the template. The primer is hybridized with a target DNA including a polymorphic site, and initiates amplification of an allele having complete homology to the primer. The primer is used as a primer pair with the other primer hybridized at the opposite side. Amplification is performed from the two primers, indicating that there is a specific allele. The primer of the present embodiment may be preferably a primer selected from the group consisting of SEQ ID Nos. 6 to 11, SEQ ID Nos. 14 to 17, and SEQ ID Nos. 36 to 41.

Preferably, the primer may be a primer pair selected from the group consisting of: a primer pair represented by SEQ ID Nos. 6 and 7 for detecting a polymorphic site of the 44^{th} base of SEQ ID No. 1; a primer pair represented by SEQ ID Nos. 8 and 9 or SEQ ID Nos. 10 and 11 for detecting a polymorphic site of the 216^{th} base of SEQ ID No. 1; a primer pair represented by SEQ ID Nos. 14 and 15 or SEQ ID Nos. 16 and 17 for detecting a polymorphic site of the 519^{th} base of SEQ ID No. 1; a primer pair represented by SEQ ID Nos. 36 and 37 for detecting a polymorphic site of the 2028^{th} base of SEQ ID No. 1; a primer pair represented by SEQ ID Nos. 38 and 39 for detecting a polymorphic site of the 2133^{th} base of SEQ ID No. 1; and a primer pair represented by SEQ ID Nos. 40 and 41 for detecting a polymorphic site of the 2247^{th} base of SEQ ID No. 1.

Furthermore, the present invention provides a microarray for the diagnosis of hepatocellular carcinoma, comprising one or more polynucleotides, which are TGFβRIII (transforming growth factor receptor III) polynucleotides represented by SEQ ID No. 1, selected from the group consisting of: a polynucleotide comprising contiguous 20 to 100 DNA sequences with C or T at the 44^{th} base of SEQ ID No. 1; a polynucleotide comprising contiguous 20 to 100 DNA sequences with A or G at the 216^{th} base of SEQ ID No. 1; a polynucleotide comprising contiguous 20 to 100 DNA sequences with A or G at the 519^{th} base of SEQ ID No. 1; a polynucleotide comprising contiguous 20 to 100 DNA sequences with T or C at the 2028^{th} base of SEQ ID No. 1; a polynucleotide comprising contiguous 20 to 100 DNA sequences with G or A at the 2133^{th} base of SEQ ID No. 1; a polynucleotide comprising contiguous 20 to 100 DNA sequences with T or C at the 2247^{th} base of SEQ ID No. 1; and a complementary polynucleotide thereof.

The microarray may include a DNA or RNA polynucleotide. The microarray has the same structure as a conventional microarray, except that it includes the polynucleotide of the present invention.

The method of preparing a microarray by immobilizing a probe polynucleotide on a substrate is well known in the art.

The “probe polynucleotide” is a hybridization probe, which is an oligonucleotide capable of binding specifically to a complementary strand of a nucleic acid. Such a probe includes a peptide nucleic acid introduced by Nielsen et al., Science 254, 1497-1500 (1991). The probe of this invention is an allele-specific probe. When a polymorphic site is located in nucleic acid fragments derived from two members of the same species, the allele-specific probe can hybridize with the DNA fragment derived from one member but not with the DNA fragment derived from the other member. In this case, the hybridization conditions can be sufficiently strict for hybridization with only one allele by showing a significant difference in intensities of hybridization for different alleles. The probe of this invention can be used in a diagnosis method for detecting an allele, etc. The diagnosis method may be Southern blotting in which detection is performed using the hybridization of nucleic acids, or a method in which a microarray to which the probe is bound in advance is used.

The hybridization can be carried out under strict conditions, for example, in a salt concentration of 1 M or less and at a temperature of 25° C. or higher. For example, 5×SSPE (750 mM NaCl, 50 mM Na Phosphate, 5 mM EDTA, pH 7.4) and 25 to 30° C. may be suitable conditions for the allele-specific probe hybridization.

The immobilization of the probe polynucleotide associated with the diagnosis of hepatocellular carcinoma on a substrate can also be easily performed using a conventional technology. Also, the hybridization of nucleic acid on the microarray and the detection of the hybridization result are well known in the art. For example, the nucleic acid sample is labeled with a fluorescent material, for example, a labeling material capable of generating detectable signals including Cy3 and Cy5, and then is hybridized on the microarray, followed by detecting signals generated from the labeling material.

Further, in order to provide information required for the diagnosis of hepatocellular carcinoma, the present invention provides a method for detecting a diagnostic marker for hepatocellular carcinoma. The above detection method may be performed by detecting the presence or absence of a marker protein or nucleic acid of the present invention in a biological sample.

More preferably, as for the method for detecting a diagnostic marker for hepatocellular carcinoma according to the present invention, there is provided a method for detecting a polymorphism (44C/T) of the 44^{th} base of SEQ ID No. 1, a polymorphism (216A/G) of the 216^{th} base of SEQ ID No. 1, a polymorphism (519A/G) of the 519^{th} base of SEQ ID No. 1, a polymorphism (2028T/C) of the 2028^{th} base of SEQ ID No. 1, a polymorphism (2133G/A) of the 2133^{th} base of SEQ ID No. 1, or a polymorphism (2247T/C) of the 2247^{th} base of SEQ ID No. 1 from a TGFβRIII (transforming growth factor receptor III) in a polynucleotide represented by SEQ ID No. 1 by base sequence analysis from patient samples.

The base sequence analysis may be performed by sequencing analysis, hybridization by microarray, allele specific PCR, dynamic allele-specific hybridization (DASH), PCR extension assay, or TaqMan technique.

In one embodiment of the present invention, Furthermore, the method for detecting a diagnostic marker for hepatocellular carcinoma according to the present invention comprises the steps of: (a) obtaining a nucleic acid sample from a specimen; (b) amplifying one or more polymorphic sites selected from the group consisting of the 44^{th} base, 216^{th} base, 519^{th} base, 2028^{th} base, 2133th base, and 2247^{th} base of SEQ ID No. 1 of a TGFβRIII (transforming growth factor receptor III) polynucleotide represented by SEQ ID No. 1; and (c) determining by analysis of the amplified DNA sequence whether the 44^{th} base of the polynucleotide of SEQ ID No. 1 is C or T, whether the 216^{th} base of the polynucleotide of SEQ ID No. 1 is A or G, whether the 519^{th} base of the polynucleotide of SEQ ID No. 1 is A or G, whether the 2028^{th} base of the polynucleotide of SEQ ID No. 1 is T or C, whether the 2133^{th} base of the polynucleotide of SEQ ID No. 1 is G or A, or whether the 2247^{th} base of the polynucleotide of SEQ ID No. 1 is T or C.

Therefore, the method for detecting a diagnostic marker for hepatocellular carcinoma according to the present invention allows it to predict and diagnose susceptibility to hepatocellular carcinoma or the development of hepatocellular carcinoma by identifying the aforementioned polymorphic bases.

The step (a) of obtaining genomic DNA from a specimen may be carried out according to the conventional DNA isolation method. The step (b) of amplifying a polymorphic site may be carried out according to the conventional amplification method. For example, a target nucleic acid may be amplified using a PCR method and obtained through a purification process. In addition, ligase chain reaction (LCR) (Wu and Wallace, Genomics 4, 560 (1989), Landegren, et al., Science 241, 1077 (1988)), transcription amplification (Kwok, et al., Proc. Natl. Acad. Sci. USA 86, 1173 (1989)), self-sustained sequence replication (Guatelli, et al., Proc. Natl. Acad. Sci. USA 87, 1874 (1990)), and nucleic acid-based sequence amplification (NASBA) may be used herein. The determination in the step (c) may be carried out using sequencing analysis, hybridization by microarray, allele specific PCR, dynamic allele-specific hybridization (DASH), PCR extension assay, PCR-RFLP assay, or TaqMan technique.

In the present invention, a method for predicting or diagnosing susceptibility to hepatocellular carcinoma or the development of hepatocellular carcinoma preferably comprises the steps of: obtaining a nucleic acid sample from a specimen; amplifying one or more polymorphic sites selected from the group consisting of the 44^{th} base, 216^{th} base, 519^{th} base, 2028^{th} base, 2133th base, and 2247^{th} base of SEQ ID No. 1 of a TGFβRIII (transforming growth factor receptor III) polynucleotide represented by SEQ ID No. 1; and determining by analysis of the amplified DNA sequence whether the 44^{th} base of the polynucleotide of SEQ ID No. 1 is C or T, whether the 216^{th} base of the polynucleotide of SEQ ID No. 1 is A or G, whether the 519^{th} base of the polynucleotide of SEQ ID No. 1 is A or G, whether the 2028^{th} base of the polynucleotide of SEQ ID No. 1 is T or C, whether the 2133^{th} base of the polynucleotide of SEQ ID No. is G or A, or whether the 2247^{th} base of the polynucleotide of SEQ ID No. 1 is T or C.

Moreover, the method for amplifying a polymorphic site may be performed using a primer pair selected from the group consisting of: a primer pair represented by SEQ ID Nos. 6 and 7 capable of detecting a polymorphic site of the 44^{th} base of SEQ ID No. 1; a primer pair represented by SEQ ID Nos. 8 and 9 or SEQ ID Nos. 10 and 11 capable of detecting a polymorphic site of the 216^{th} base of SEQ ID No. 1; a primer pair represented by SEQ ID Nos. 14 and 15 or SEQ ID Nos. 16 and 17 capable of detecting a polymorphic site of the 519^{th} base of SEQ ID No. 1; a primer pair represented by SEQ ID Nos. 36 and 37 capable of detecting a polymorphic site of the 2028^{th} base of SEQ ID No. 1; a primer pair represented by SEQ ID Nos. 38 and 39 capable of detecting a polymorphic site of the 2133^{th} base of SEQ ID No. 1; and a primer pair represented by SEQ ID Nos. 40 and 41 capable of detecting a polymorphic site of the 2247^{th} base of SEQ ID No. 1.

The method for predicting or diagnosing susceptibility to hepatocellular carcinoma or the development of hepatocellular carcinoma may further comprise the step of determining that the risk of developing hepatocellular carcinoma is high if, in the polynucleotide of SEQ ID No. 1, the 44^{th} base is T, the 216^{th} base is G, the 519^{th} base is A, the 2028^{th} base is T, the 2133th base is G, or the 2247^{th} base is C. The determination of the risk of development can be carried out based on the frequency of polymorphisms found in carcinoma tissue in accordance with one embodiment of the present invention to be discussed below.

The invention will now be further explained in the following examples. These examples are only intended to illustrate the invention and should in no way be considered to limit the scope of the invention.

Unless otherwise indicated, nucleic acids are written left to right in 5′ to 3′ orientation. Numeric ranges recited within the specification are inclusive of the numbers defining the range and include each integer or any non-integer fraction within the defined range.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

### Example 1

### Preparation of DNA Samples

<1-1> Tissue Samples

Sixty-seven frozen HCCs and their corresponding background normal tissue samples obtained from 67 patients at resection were evaluated. Approval was obtained from the Institutional Review Board of College of Medicine, The Catholic University of Korea and informed consent was obtained beforehand in accord with the requirements of the Declaration of Helsinki. There was no evidence of familial cancer in any of the patients. The ages of the patients ranged from 4-74 (average 55 years) and there were 48 men and 19 women. The background liver demonstrated the presence of cirrhosis in 24 (35.8%) cases, chronic hepatitis in 40 (59.7) and no specific change in 3 (4.5%). HBV was detected in 51 (76.1%) and HCV in 4 patients (6%). Histologically, 1, 39, and 27 samples were Edmonson grades I, II, and III, respectively.

<1-2> Cell Culture

Ten HCC cell lines, HepG2, Hep3B, PLC/PRF/5, SNU-182, SNU-354, SNU-368, SNU-387, SNU-423, SNU-449, and SNU-475 were cultured in RPMI-1640 medium (Lonza, Walkersville, Md., USA) containing 10% fetal bovine serum (Lonza) and 1% penicillin/streptomycin (Invitrogen, Carlsbad, Calif., USA). An immortalized normal liver cell line, THLE3, was maintained in BEBM medium (Lonza) supplemented with the BEGM Bullet kit and 10% fetal bovine serum (Lonza).

<1-3> DNA Extraction

Frozen tissue samples were ground to a fine powder in liquid nitrogen and this powder was incubated overnight in 500 μl of lysis buffer (5 mM Tris-Cl pH 8.0, 20 mM EDTA, 0.5% Triton X-100) containing 500 μg/ml of proteinase K (Takara Bio Inc., Shiga, Japan) at 50° C. Eleven cell lines were lyzed with the same buffer in the presence of 100 μg/ml of proteinase K (Takara Bio Inc). Phenol: chloroform: isoamyl alcohol (25:24:1) solution (Sigma-Aldrich Corp., St. Louis, Mo., USA) was then added to each lysate and after centrifugation (15,000 rpm, 4° C., 30 min), phenol: chloroform: isoamyl alcohol (25:24:1) solution was added to the supernatants. DNA was precipitated with ethanol at −70° C. and washed with 70% ethanol. The dried pellets were resuspended with 1×TE buffer (10 mM Tris-Cl pH 8.0, 1 mM EDTA).

### Example 2

### Quantitative RT-PCR Analysis

Total RNA was isolated from frozen tissues using TRIzol reagent (Invitrogen) according to the manufacturer's instructions. cDNA was synthesized with the transcriptor first-strand cDNA synthesis kit (Roche Applied Science, Indianapolis, Ind., USA). Real-time PCR analysis was performed in a total volume of 12.5 μl mixture containing 3 μm of 50-fold diluted cDNA, 0.4 μM of sense and antisense primers and 6.25 μl of iQ™ SYBR® Green supermix (Bio-Rad Laboratories, Hercules, Calif., USA). To normalize differences in the amount of total cDNA added to each reaction, glyceraldehydes 3-phosphate dehydrogenase (GAPDH) gene expression was used as an endogenous control. The reaction mixture was denatured for 3 min at 95° C. and incubated for 40 cycles (denaturing for 15 sec at 95° C., annealing for 20 sec at 62° C. and extension for 20 sec at 72° C.) The primers used in the experiment were as shown below. The PCR was monitored in real-time using an iQ™-5 (Bio-Rad Laboratories) that allowed determination of the threshold cycle (Ct) at the time exponential amplification of the PCR products began. The average Ct, from duplicate assays, was used for further calculation and GAPDH-normalized gene expression was determined using the relative quantification method described as the following:

Relative expression levels normalized to GAPDH=2−(TGFβRIII Ct−GAPDH Ct)×100.

The results are expressed as the mean value of duplicates.

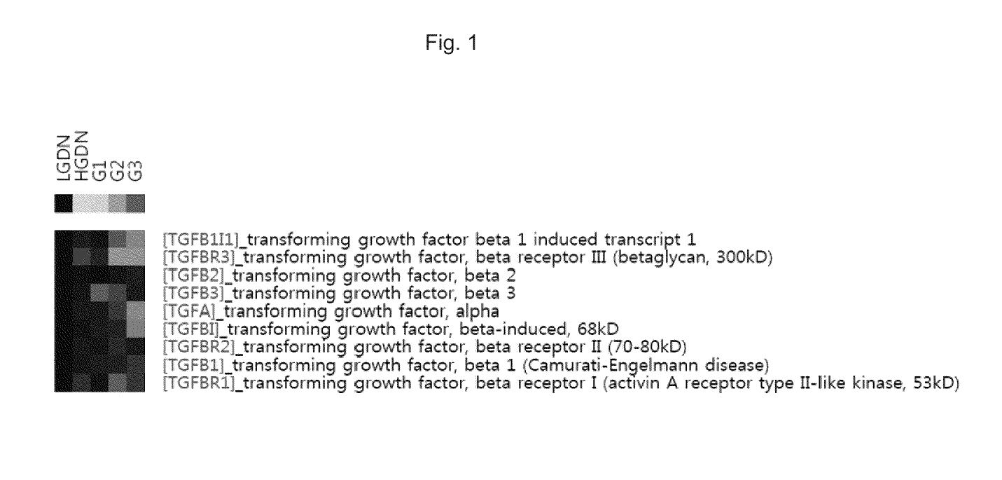

Also, it has been already known that TGF-β and the TGF-β receptor family were differentially regulated during the progression of HCC. TGFβRIII was gradually down-regulated from a dysplastic nodule, a precancerous region of the HCC, to Edmonson grades of HCC and over HCC (FIG. 1).

TABLE 1
Primer Sequences

| | | SEQ ID |
|---|---|---|
| Primer Name | Base Sequence | No. |
| TGFβRIII sense | 5′-ACCGTGATGGGCATTGCGTTTGCA-3′ | 2 |
| TGFβRIII anti- | 5′-GTGCTCTGCGTGCTGCCGATGcTGT-3′ | 3 |
| sense | | |
| GAPDH sense | 5′-ACCAGGTGGTCTCCTCTGAC-3′ | 4 |
| GAPDH anti- | 5′-TGCTGTAGCCAAATTCGTTG-3′ | 5 |
| sense | | |

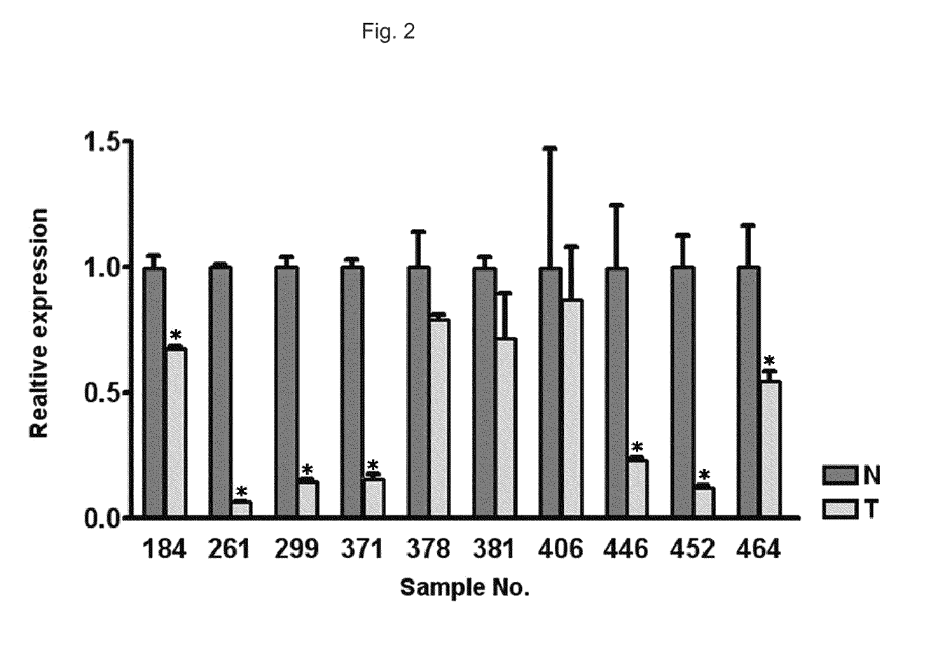

As a result, 7 out of 10 selected HCCs had significantly reduced expression compared to the corresponding normal tissues; the remaining three had mean values of down-regulation (FIG. 2).

### Example 3

### SSCP (Single-Strand Conformation Polymorphism) and DNA Sequencing Analysis

Genomic DNA samples from cell lines and HCC tissues of Example 1 were amplified with 18 sets of primers covering all of the coding regions of the TGFβRIII gene of the following Table 2. The following primer sequences corresponding to E2F to E17R were named SEQ ID Nos 6 to 47. The PCRs were performed under conditions with 10 μm reaction mixtures containing 10 ng of template DNA, 0.1 mM of each deoxynucleotide triphosphate (Promega, Madison, Wis., USA), 1.5 mM of MgCl₂, 0.5 unit of Ampli Taq gold polymerase, 1 μl of 10× buffer (Perkin-Elmer, Foster City, Calif., USA) and 1 μCi of [³²P]dCTP (Amersham, Buckinghamshire, UK). The reaction mixtures were initially denatured for 12 min at 95° C., then subjected to 40 amplification cycles (denaturing for 30 sec at 95° C., annealing for 30 sec at 49-54C and extension for 30 sec at 72° C.) and a final extension for 5 min at 72° C. After amplification, the PCR products were denatured for 5 min at 95° C., in a 1:1 dilution of sample buffer containing 98% formamide/5 mmol/l NaOH and loaded onto an SSCP gel (Mutation Detection Enhancement, FMC BioProduct, Rockland, Me., USA) with 10% glycerol. After electrophoresis, the gels were transferred to 3 mM Whatman paper and dried. Autoradiography was performed using Kodak X-OMAT film (Eastman Kodak, Rochester, N.Y., USA). To detect mutations, the DNAs showing mobility shifts were excised from the dried SSCP gels and re-amplified over 40 cycles using the same primer sets. After electrophoresis in 2% agarose gels, the PCR products were eluted from the gels with the Qiaquick gel extraction kit (Qiagen, Valencia, Calif., USA) following the manufacturer's instructions. Sequencing of the PCR products was performed by COSMO Co., Ltd (Seoul, Korea). The results were shown in the following Table 3.

TABLE 2
Primers for amplifying TGFβRIII gene

| | | Product |
|---|---|---|
| Exon | Nucleotide Sequence | size (bp) |
| E2F | 5′-CTGAAGTGACTGGACGAGA-3′ | 200 |
| | (SEQ ID NO: 6) | |
| E2R | 5′-CCTGGGTAACAGAGTGAAAC-3′ | |
| | (SEQ ID NO: 7) | |
| E3-1F | 5′-TGATCACCCTTGCCCCTTTG-3′ | 229 |
| | (SEQ ID NO: 8) | |
| E3-1R | 5'-TGCAGTGCGGAGATTCAGGA-3′ | |
| | (SEQ ID NO: 9) | |
| E3-2F | 5′-GTTTTGTCAGGCTGTGC-3′ | 199 |
| | (SEQ ID NO: 10) | |
| E3-2R | 5'-GTTGAACCCCAGAAGAGA-3′ | |
| | (SEQ ID NO: 11) | |
| E4F | 5′-TTTCTGCCCTCTTTCTGTT-3′ | 217 |
| | (SEQ ID NO: 12) | |
| E4R | 5′-CCATTATGTCCTTGTGCTAAG-3′ | |
| | (SEQ ID NO: 13) | |
| E5-1F | 5′-AGGTTCGATTTACAAGCA-3′ | 202 |
| | (SEQ ID NO: 14) | |
| E5-1R | 5′-TTAACAGATGTTCVATTTCCA-3′ | |
| | (SEQ ID NO: 15) | |
| E5-2F | 5′-CAGCAAACTTCTCCTTGA-3′ | 200 |
| | (SEQ ID NO: 16) | |
| E5-2R | 5′-ATTGCCTGTCATAAATCAGT-3′ | |
| | (SEQ ID NO: 17) | |
| E6F | 5′-CCTCAGTGGTTTGACAGATT-3′ | 245 |
| | (SEQ ID NO: 18) | |
| E6R | 5′-TCATCTCTTGTCACACTCACA-3′ | |
| | (SEQ ID NO: 19) | |
| E7F | 5′-AACTTTCTGGCATGTAGGTC-3′ | 217 |
| | (SEQ ID NO: 20) | |
| E7R | 5′-GACATGCTCCACCAACTT-3′ | |
| | (SEQ ID NO: 21) | |
| E8-1F | 5′-ATTTTAGACTCATGAGTGATTT-3′ | 188 |
| | (SEQ ID NO: 22) | |
| E8-1R | 5'-GCCATTGTGTATGAAGTTAT-3′ | |
| | (SEQ ID NO: 23) | |
| E8-2F | 5′-CCAAATCAATAAGAGATGAC-3′ | 213 |
| | (SEQ ID NO: 24) | |
| E8-2R | 5'-GAAATGACAGTTCCTCACT-3′ | |
| | (SEQ ID NO: 25) | |
| E9-1F | 5′-GGCCTGGCATCAAACACT-3′ | 235 |
| | (SEQ ID NO: 26) | |
| E9-1R | 5′-GAGCCCATCTTCTCCCTCTT-3′ | |
| | (SEQ ID NO: 27) | |
| E9-2F | 5′-CCGTTTCCTTTCCCAGAT-3′ | 248 |
| | (SEQ ID NO: 28) | |
| E9-2R | 5′-TAGCCTCTCTTCCCTCCTG-3′ | |
| | (SEQ ID NO: 29) | |
| E10F | 5′-ACAGAACTGCCTGTGGG-3′ | 231 |
| | (SEQ ID NO: 30) | |
| E10R | 5′-CAAAGCTTTGTTCTGGAAAA-3′ | |
| | (SEQ ID NO: 31) | |
| E11F | 5′-AGGCAGAACCAAACACA-3′ | 233 |
| | (SEQ ID NO: 32) | |
| E11R | 5′-ACCCCCTACTGATAACAAAC-3′ | |
| | (SEQ ID NO: 33) | |
| E12F | 5′-CCTGTGGGTTGTTATTTCC-3′ | 208 |
| | (SEQ ID NO: 34) | |
| E12R | 5′-AAGGTCAAGGCTAACTTTCAG-3′ | |
| | (SEQ ID NO: 35) | |
| E13-1F | 5′-TTGTGCCTAAAGTGAAAGTG-3′ | 243 |
| | (SEQ ID NO: 36) | |
| E13-1R | 5′-TCAGCTTGCGGGATAG-3′ | |
| | (SEQ ID NO: 37) | |
| E13-2F | 5′-GAAATTCTACAGTCCCAAGA-3′ | 215 |
| | (SEQ ID NO: 38) | |
| E13-2R | 5′-CTAAAAATGCCAAAATAACC-3′ | |
| | (SEQ ID NO: 39) | |
| E14F | 5′-CCCTGATTCTGTGCTTTGT-3′ | 180 |
| | (SEQ ID NO: 40) | |
| E14R | 5′-TCTGATCGTGCCTCCC-3′ | |
| | (SEQ ID NO: 41) | |
| E15F | 5′-GTTTCTGCTGAGACTTTGAT-3′ | 164 |
| | (SEQ ID NO: 42) | |
| E15R | 5′-CCCAGGAGGTTTTATTTC-3′ | |
| | (SEQ ID NO: 43) | |
| E16F | 5′-TGATGCAGACTAACCAAAA-3′ | 196 |
| | (SEQ ID NO: 44) | |
| E16R | 5′-AAGCTGTTCACCAACTCTTA-3′ | |
| | (SEQ ID NO: 45) | |
| E17F | 5′-TGCGTCTTTCTCTGACTCTG-3′ | 219 |
| | (SEQ ID NO: 46) | |
| E17R | 5′-TGGCAGTAGCTGAGCTGA-3′ | |
| | (SEQ ID NO: 47) | |

TABLE 3

| | | | | | | Frequency | |
|---|---|---|---|---|---|---|---|
| Exon | Nucleotide | Codon | Amino Acid | Type | SNP ID | in tissues | Cell line |
| 2 | C44T | TCC→ TTC | S16F | Missense | rs1805110 | TCC (1/67) | |
| | | | | (non-synonymous) | | TTC (66/67) | |
| 3 | A215G | GCA→GCG | A72A | Silent | rs2810904 | GCA (20/67) | THLE3 |
| | | | | (synonymous) | | GCG (47/67) | Hep1B |
| | | | | | | | PLC/PRF/5 |
| | | | | | | | SNU-354 |
| | | | | | | | SNU-367 |
| | | | | | | | SNU-423 |
| | | | | | | | SNU-476 |
| 5 | A516G | TCA→ TCG | S173S | Silent | rs2306886 | TCA (47/87) | SNU-448 |
| | | | | (synonymous) | | TCG (20/87) | |
| 11 | T2029C | TTT→TTC | F679F | Silent | rs1805113 | TTT (56/67) | HepG2 |
| | | | | (synonymous) | | TTC (11/67) | SNU-182 |
| | G2113A | ACC→ACA | T711T | Silent | Unknown | ACG (66/67) | |
| | | | | (synonymous) | | ACA (1/67) | |
| 14 | T2247C | ACT→ACC | T748T | Silent | rs284878 | ACT (18/67) | THLE3 |
| | | | | (synonymous) | | ACC (49/67) | Hep3B |
| | | | | | | | HepG2 |
| | | | | | | | PLC/PRF/5 |
| | | | | | | | SNU-182 |
| | | | | | | | SNU-387 |
| | | | | | | | SNU-423 |
| | | | | | | | SNU-449 |

The results, as shown in Table 3, showed no significant mutations in any of the coding regions analyzed. However, we observed six polymorphisms in five exons. Among these, there was one novel polymorphism, not previously identified, located in exon 13 (G2133A)

Also, the present invention revealed, for the first time, that the identified six polymorphisms were present with a high frequency in hepatocellular carcinoma.

Accordingly, the present inventors have found that the six polymorphisms found in the present invention can be used as novel markers for the diagnosis of hepatocellular carcinoma.

### Example 4

### LOH (Loss of Heterozygosity) Analysis

The tumor and the corresponding normal DNA were amplified with the D1S188, D1S406, D1S435, and D1S2804 markers. PCR was performed in 10 μl reaction mixtures containing 10 ng of template DNA, 0.1 mM of each deoxynucleotide triphosphate (Promega, Madison, Wis., USA), 1.5 mM of MgCl₂, 0.5 unit of Ampli Taq gold polymerase, 1 μl of 10× buffer (Perkin-Elmer, Foster City, Calif., USA) and 1 μCi of [³²P]dCTP (Amersham, Buckinghamshire, UK). The PCR products were then denatured and electrophoresed in 6% polyacrylamide gel containing 7 M urea. After electrophoresis, the gels were transferred to 3 mM Whatman paper and dried. Autradiography was performed using Kodak X-OMAT film (Eastman Kodak). Identification of complete absence of one allele, in the tumor DNA of informative cases, by direct visualization, was considered LOH.

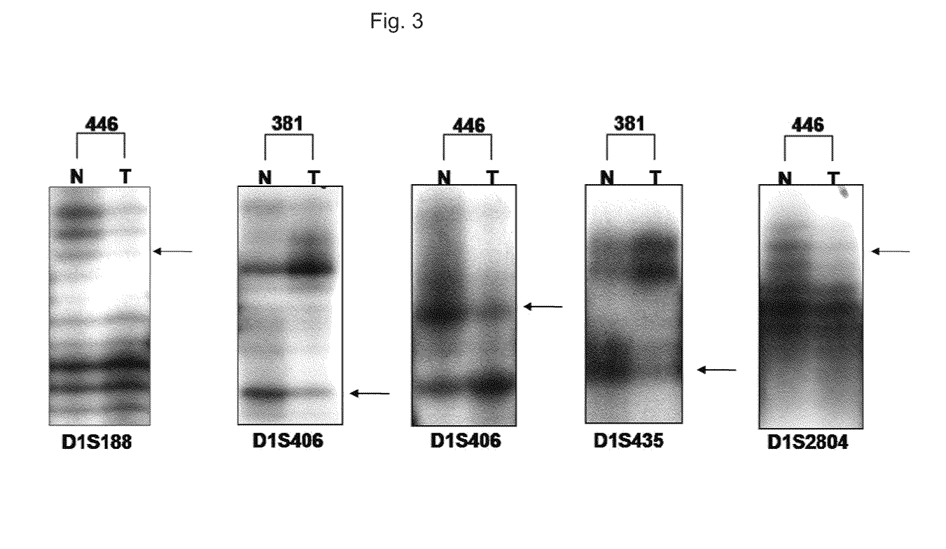

As a result of the analysis, allelic loss at the TGFβRIII locus was studied with the microsatellite markers D1S188, D1S406, D1S435, and D1S2804, in ten normal and tumor sample sets analyzed by qRT-PCR. As shown in FIG. 3, LOH was observed in two samples. Based on this, the present inventors found that loss of heterozygosity occurs at a low frequency in the TGFβRIII gene of hepatocellular carcinoma tissue.

What is claimed is:

1. A polymorphic marker for the diagnosis of hepatocellular carcinoma, comprising one or more polynucleotides, which are TGFβRIII (transforming growth factor receptor III) polynucleotides represented by SEQ ID No. 1, selected from the group consisting of: a polynucleotide comprising contiguous 20 to 100 DNA sequences with C or T at the 44^{th} base of SEQ ID No. 1; a polynucleotide comprising contiguous 20 to 100 DNA sequences with A or G at the 216^{th} base of SEQ ID No. 1; a polynucleotide comprising contiguous 20 to 100 DNA sequences with A or G at the 519^{th} base of SEQ ID No. 1; a polynucleotide comprising contiguous 20 to 100 DNA sequences with T or C at the 2028^{th} base of SEQ ID No. 1; a polynucleotide comprising contiguous 20 to 100 DNA sequences with G or A at the 2133^{th} base of SEQ ID No. 1; a polynucleotide comprising contiguous 20 to 100 DNA sequences with T or C at the 2247^{th} base of SEQ ID No. 1; and a complementary polynucleotide thereof.

2. The marker of claim 1, wherein the 44^{th} base of SEQ ID No. 1 is located in exon 2 of the TGFβRIII gene, the 216^{th} base of SEQ ID No. 1 is located in exon 3 of the TGFβRIII gene, the 519^{th} base of SEQ ID No. 1 is located in exon 5 of the TGFβRIII gene, the 2028^{th} and 2123th bases of SEQ ID No. 1 are located in exon 13 of the TGFβRIII gene, and the 2247^{th} base of SEQ ID No. 1 is located in exon 14 of the TGFβRIII gene.

3. The marker of claim 1, wherein the hepatocellular carcinoma is human hepatocellular carcinoma.

4. A diagnostic composition for hepatocellular carcinoma, the composition comprising a primer for amplifying a polynucleotide, which is a TGFβRIII (transforming growth factor receptor III) polynucleotide represented by SEQ ID No. 1, the polynucleotide comprising: a polymorphic site of the 44^{th} base of SEQ ID No. 1; a polymorphic site of the 216^{th} base of SEQ ID No. 1; a polymorphic site of the 519^{th} base of SEQ ID No. 1; a polymorphic site of the 2028^{th} base of SEQ ID No. 1; a polymorphic site of the 2133^{th} base of SEQ ID No. 1; or a polymorphic site of the 2247^{th} base of SEQ ID No. 1.

5. The diagnostic composition of claim 4, wherein the primer is selected from the group consisting of:
a primer pair represented by SEQ ID Nos. 6 and 7 for detecting a polymorphic site of the 44^{th} base of SEQ ID No. 1;
a primer pair represented by SEQ ID Nos. 8 and 9 or SEQ ID Nos. 10 and 11 for detecting a polymorphic site of the 216^{th} base of SEQ ID No. 1;
a primer pair represented by SEQ ID Nos. 14 and 15 or SEQ ID Nos. 16 and 17 for detecting a polymorphic site of the 519^{th} base of SEQ ID No. 1;
a primer pair represented by SEQ ID Nos. 36 and 37 for detecting a polymorphic site of the 2028^{th} base of SEQ ID No. 1;
a primer pair represented by SEQ ID Nos. 38 and 39 for detecting a polymorphic site of the 2133^{th} base of SEQ ID No. 1; and
a primer pair represented by SEQ ID Nos. 40 and 41 for detecting a polymorphic site of the 2247^{th} base of SEQ ID No. 1.

6. A microarray for the diagnosis of hepatocellular carcinoma, comprising the polynucleotides of claim 1.

7. A diagnostic kit for hepatocellular carcinoma comprising the diagnostic composition of claim 4.

8. A method for detecting a polymorphism (44C/T) of the 44^{th} base of SEQ ID No. 1, a polymorphism (216A/G) of the 216^{th} base of SEQ ID No. 1, a polymorphism (519A/G) of the 519^{th} base of SEQ ID No. 1, a polymorphism (2028T/C) of the 2028^{th} base of SEQ ID No. 1, a polymorphism (2133G/A) of the 2133^{th} base of SEQ ID No. 1, or a polymorphism (2247T/C) of the 2247^{th} base of SEQ ID No. 1 from a TGFβRIII (transforming growth factor receptor III) in a polynucleotide represented by SEQ ID No. 1 by base sequence analysis from patient samples in order to provide information required for the diagnosis of hepatocellular carcinoma.

9. The method of claim 8, wherein the base sequence analysis is performed by sequencing analysis, hybridization by microarray, allele specific PCR, dynamic allele-specific hybridization (DASH), PCR extension assay, or TaqMan technique.

10. A method for predicting or diagnosing hepatocellular carcinoma, the method comprising the steps of:
obtaining a nucleic acid sample from a specimen;
amplifying one or more polymorphic sites selected from the group consisting of the 44^{th} base, 216^{th} base, 519^{th} base, 2028^{th} base, 2133th base, and 2247^{th} base of SEQ ID No. 1 of a TGFβRIII (transforming growth factor receptor III) polynucleotide represented by SEQ ID No. 1; and
determining by analysis of the amplified DNA sequence whether the 44^{th} base of the polynucleotide of SEQ ID No. 1 is C or T, whether the 216^{th} base of the polynucleotide of SEQ ID No. 1 is A or G, whether the 519^{th} base of the polynucleotide of SEQ ID No. 1 is A or G, whether the 2028^{th} base of the polynucleotide of SEQ ID No. 1 is T or C, whether the 2133^{th} base of the polynucleotide of SEQ ID No. 1 is G or A, or whether the 2247^{th} base of the polynucleotide of SEQ ID No. 1 is T or C.

11. The method of claim 10, wherein the amplification of a polymorphic site is performed using a primer pair selected from the group consisting of: a primer pair represented by SEQ ID Nos. 6 and 7 capable of detecting a polymorphic site of the 44^{th} base of SEQ ID No. 1; a primer pair represented by SEQ ID Nos. 8 and 9 or SEQ ID Nos. 10 and 11 capable of detecting a polymorphic site of the 216^{th} base of SEQ ID No. 1; a primer pair represented by SEQ ID Nos. 14 and 15 or SEQ ID Nos. 16 and 17 capable of detecting a polymorphic site of the 519^{th} base of SEQ ID No. 1; a primer pair represented by SEQ ID Nos. 36 and 37 capable of detecting a polymorphic site of the 2028^{th} base of SEQ ID No. 1; a primer pair represented by SEQ ID Nos. 38 and 39 capable of detecting a polymorphic site of the 2133^{th} base of SEQ ID No. 1; and a primer pair represented by SEQ ID Nos. 40 and 41 capable of detecting a polymorphic site of the 2247^{th} base of SEQ ID No. 1.

12. The method of claim 10, further comprising the step of determining that the risk of developing hepatocellular carcinoma is high if, in the polynucleotide of SEQ ID No. 1, the 44^{th} base is T, the 216^{th} base is G, the 519^{th} base is A, the 2028^{th} base is T, the 2133th base is G, or the 2247^{th} base is C.
